(19) United States
(12) Patent Application Publication
(10) Pub. No.: US 2006/0293386 A1
(43) Pub. Date: Dec. 28, 2006

(54) Method for treating cancer

(76) Inventor: Tsuneo Ozeki, Kitakyushu-city (JP)

Correspondence Address: JORDAN AND HAMBURG LLP, 122 EAST 42ND STREET, SUITE 4000, NEW YORK, NY, 10168, US

(21) Appl. No.: 11/256,747

(22) Filed: Oct. 24, 2005

(30) Foreign Application Priority Data
Jun. 24, 2005 (JP) 2005-185776

(51) Int. Cl. A61K 31/235

(52) U.S. Cl. 514/533

(57) ABSTRACT

It is an object of the present invention to provide a method for cancer treatment to suppress cancer, specifically hepato cellular carcinoma. There is discovered effective suppression of the progression of hepatocellular carcinoma with gabexate mesilate, a non-anticancer drug that has not been used as an anticancer drug. Gabexate mesilate in the amount of 100-300 mg dissolved in 150-300 ml of physiological saline or of 5% glucose is administered by intravenous drip infusion. The administration can be carried out noninvasively with little or no pain to patients. Concomitant use with embolization strongly suppresses hepatocellular carcinoma.

### BACKGROUND OF THE INVENTION

(1) Field of the invention

The present invention relates to a method for treating cancer. More specifically, the invention relates to a method for treating cancer that noninvasively suppresses cancer, especially hepato cellular carcinoma, without causing pain. The present invention is a treatment method that uses a drug that does not belong to a category of so-called anticancer drugs.

(2) Description of Related Art

As is commonly known, hepatitis C, which has attracted attention for a higher frequency of hepatocellular carcinoma (hereinafter referred to as “HCC”) development, is a hepatitis caused by infection with the hepatitis C virus (HCV), the causative agent thereof. Hepatitis C is presumed to be almost incurable with antibodies, unlike hepatitis B, in which hepatitis B virus antigens are eliminated by antibodies (seroconversion) to cure the hepatitis. There are asymptomatic carriers of hepatitis C, besides those with symptoms such as acute hepatitis, chronic hepatitis, and liver cirrhosis. In hepatitis C, acute hepatitis cannot be noticed by patients themselves, and most hepatitides are diagnosed as chronic hepatitis that lead to further hepatic dysfunctions and progression to liver cirrhosis. Interferon (IFN) therapy is used extensively for medication of chronic hepatitis C. In Japan, most HCV genotypes are type II or type III. IFN is effective in type III, but effectiveness is bad in type II. However, IFN is effective in type II with many mutations in NS5A.

The frequency of progression from chronic hepatitis or liver cirrhosis to HCC is estimated to be approximately 2.5 fold higher in hepatitis C than in hepatitis B. In Japan, unlike in Europe and the United States, it is reported that HCC derives more from liver cirrhosis than from chronic hepatitis.

Currently, HCC therapy includes surgical and nonsurgical treatments. Surgical treatment includes hepatectomy (liver lobectomy and segmental resection) of HCC lesions.

Meanwhile, nonsurgical treatment includes percutaneous ethanol injection therapy (hereinafter referred to as “PEIT”), which causes necrosis by ultrasound-guided injection of ethanol into the tumor, percutaneous radiofrequency ablation (hereinafter referred to as “RFA”), which degenerates and makes necrosis cancer tissue by coagulation using Joule heat by radiofrequency alternating current and tissue impedance passed through by the ultrasound-guided electrode inserted into the tumor; and presently the most frequently used transcatheter arterial embolization (hereinafter referred to as “TAE”), which administers an anticancer drug (such as mitomycin and adriamycin) into the tumor by a catheter inserted through an artery to the vicinity of liver tumors, and moreover, which occludes tumor-feeding artery (tumor vessels) by spongel infusion.

BRIEF SUMMARY OF THE INVENTION

However, in surgical treatment, namely a surgical operation, the liver cirrhosis must belong to class A according to the Child-Pugh classification, and in the case of class B, residual cancer tissues after partial resection or tumorectomy might recur. Also, development of liver failure and gastrointestinal hemorrhage, or possible new HCC in the residual liver might cause other problems.

Additionally, IFN therapy has been tried as HCC treatment, but its effectiveness remains uncertain. Additionally, PEIT might cause development of not only many complications such as hematoma, hemorrhage, pneumothrax, hematothorax, biliary peritonitis, and biloma, but new satellite tumors that adversely result in an increase in the number of tumors. And, reportedly the 5-year recurrence rate is 98%.

Meanwhile, RFA is superior to PEIT, but not effective against tumors in the vicinity of large vessels and its complications, are intraabdominal hemorrhage, liver abscess, and portal hematoma. TAE is conducted by inserting a catheter through an artery close to the tumor to administer an anticancer drug and infuse spongel to occlude tumor vessels. Another TAE is conducted after an average of 3-4 months. This corresponds with the time of new formation of natural bypass of tumor vessels after vascular occlusion. Therefore, TAE is repeated at intervals on average of 4-6 months. However, there is a disadvantage that the damage to the vascular intima caused by a catheter after conducting TAE 7 or 8 times might make it impossible to conduct another TAE. TAE is conducted in combination with the current hepato cellular carcinoma treatment, however, its 3.5-year survival rate is 50%. Therefore, another method for hepato cellular carcinoma treatment is required to improve the survival rate.

The present invention was made on the grounds of the above points, and intended to provide a method for treating cancer to suppress the viability of cancer and to improve the survival time of hepato cellular carcinoma patients.

The inventor has found the effective suppression of progression to HCC with gabexate mesilate, a non-anticancer drug that has not been used as an anticancer drug in the past, and completed the present invention.

In the method for treating cancer of the present invention, gabexate mesilate is administered to a cancer patient to achieve the above object.

The method for treating cancer related to the present invention exerts effects to suppress the viability of cancer.

### BRIEF DESCRIPTION OF THE DRAWINGS

FIG. 1 is a diagram illustrating the transition of AFP values when gabexate mesilate is administered;

FIG. 2 is a diagram illustrating the transition of PIVKA-II values when gabexate mesilate is administered;

FIG. 3 is a diagram illustrating the average value and standard deviations of AFP values in the GM-nonadministered HCC group; and

FIG. 4 is a diagram illustrating the average values and standard deviations of AFP values in the GM-administered HCC group with abnormal AFP values.

### DETAILED DESCRIPTION OF THE INVENTION

AFP (Alpha-fetoprotein) and PIVKA-II (isomer of prothrombin) were used as tumor markers of HCC. Elevation of these markers in HCC suggests the viability or enlargement of tumors. In general, it is often difficult to determine the shrinkage of cancer lesions by CT scanning or echo imaging immediately after conducting TAE, therefore these markers are used to determine the effects. In Japan, the conventional Alpha-feto Riabeads method has been replaced by the Chemilumi ACS-AFP method since May 2004. The former is a radiation immunoassay whose standard value is ≦20. Meanwhile, the latter is a chemiluminescence immunoassay whose standard value is ≦10. However, values measured by these two methods are almost comparable in spite of these different standard values.

Liver function tests (total bilirubin, direct bilirubin, total protein, albumin, protein fraction, GOT, GPT, TTT, ZTT, prothrombin, hepaplastin, and cholinesterase) and peripheral blood tests (erythrocyte, leukocyte, hemoglobin, hematocrit, and platelet) and others were conducted for the diagnosis of liver diseases. In addition, contrast CT and ultrasound (echo) exams were conducted for image diagnosis, and some cases were diagnosed by liver biopsy.

Blood virus markers such as HBs and HBe antigens for hepatitis B, and HCV antibody for hepatitis C (LA method) were used, and HCV RNA quantification (Amplicor method) and other tests for hepatitis C were performed. Chronic hepatitis C was determined by being positive for HCV antibody and HCV PCR quantification (Amplicor method) and negative for HBs antigen, and by being diagnosed as chronic hepatitis or liver cirrhosis by liver biopsy, however, in cases where liver biopsy cannot be performed, diagnosis of liver cirrhosis was made when four or more of the following conditions are met: albumin <4.0 g/dl, platelet count <120,000, cholinesterase <3,500 U/L, hepaplastin <70%, and prothrombin <70%, and patterns of liver cirrhosis (enlarged left lobe, shrinkage of the right lobe, and open cleavage of the liver) were confirmed by contrast CT and echo exams, meanwhile, diagnosis of chronic hepatitis was made when HBV or HCV infection and liver dysfunctions were confirmed; functional conditions of liver cirrhosis were not met; pathology of liver cirrhosis was not observed by echo and contrast CT exams, but edging off or slight nonuniformity in the liver margin was confirmed. Meanwhile, 3(three) chronic hepatitis cases were diagnosed by liver biopsy. And the Child-Pugh classification was used for the staging of liver cirrhosis.

HCC diagnosis was conducted using abdominal echo, contrast CT, CTA, CTAP, MRI and other exams. MRI was used specifically for protruded lesions of the liver surface (just beneath the dome).

Diameters of the tumors were measured mainly on contrast CT. However, it was difficult to correctly measure the tumor diameters on CT or echo exam after lipiodol administration by TAE.

HCC patients and AFP-positive (precancerous lesions or regenerating hepatocytes) patients without HCC observed on the images (on CT or echo exam) were given 100-300 mg of gabexate mesilate (hereinafter referred to as “GM”) dissolved in 150-300 ml of physiological saline or of 5% glucose by intravenous drip infusion. GM is originally a drug applicable to chronic pancreatitis, acute pancreatitis, and DIC (disseminated intravascular coagulation), however, informed consent was obtained before the administration. And in allergy cases, the starting dose was 75-100 mg and increased. The nonadministered group is GM-nonadministered HCC cases.

TABLE 1 is the transition of AFP values in 8(eight) GM-nonadministered HCC cases, including 7(seven) liver cirrhosis patients (C type, hereinafter referred to as “C”) and 1(one) chronic hepatitis patient (C). AFP values of each patient were measured by the Alpha-feto Riabeads method. Theses AFP values, measured before the treatment, increased significantly every month compared with 0 month. Meanwhile, “M” in the TABLE indicates month; 2M indicates 2 months after 0M, for example.

TABLE 1

| | Sex | Age | Diagnosis | 0 M | 1 M | 2 M | 3 M | Tumor size |
|---|---|---|---|---|---|---|---|---|
| 1 | Female | 71 | CH | 17.4 | 30.5 | 48.7 | 56.7 | S₃ Φ (Diameter) 3.3 cm |
| 2 | Female | 74 | LC | 23.6 | 28.5 | 30.6 | 34.1 | Occurred in multiple places (S₆ Φ2.3 cm and |
| | | | | | | | | others) |
| 3 | Female | 69 | LC | 14.4 | 30.8 | 42.5 | 66.9 | S₈ Φ3.8 × 1.8 cm |
| 4 | Male | 63 | LC | 43.0 | | 62.7 | 85.0 | Occurred in multiple places (S₈ Φ2.3 cm and |
| | | | | | | | | others) |
| 5 | Male | 53 | LC | 18.1 | 34.0 | 48.7 | | Occurred in multiple places (S₄ Φ3.4 × 2.7 cm and |
| | | | | | | | | others) |
| 6 | Male | 52 | LC | 13.7 | 15.3 | 20.6 | 25.2 | Occurred in multiple places (S₈ Φ1.5 cm and |
| | | | | | | | | others) |
| 7 | Male | 63 | LC | 12.9 | | 25.2 | 50.9 | S₈ Φ3 cm |
| 8 | Male | 67 | LC | 18.7 | 19.8 | 29.5 | 72.2 | S₆ Φ3 cm |

AFP: Measured by Alpha-feto Riabeads method

As shown in TABLE 1, AFP values in the GM-nonadministered cases increased every month. Statistically, the values at 2M and 3M increased relative to 0M.

TABLE 2

| | Sex | Age | Diagnosis | 0 M | 1 M | 2 M | 3 M | Tumor size |
|---|---|---|---|---|---|---|---|---|
| 1 | Male | 72 | LC | 7.2 | 5.9 | 5.8 | 4.0 | Occurred in multiple places |
| | | | | | | | | o(S₅ Φ2.5 cm and thers) |
| 2 | Female | 64 | LC | 8.5 | 12.1 | 11.6 | 13.6 | S₈ Φ2.0 cm |
| 3 | Male | 77 | LC | 6.5 | 8.9 | 6.2 | 6.7 | S₈ Φ2.0 cm |
| 4 | Male | 76 | LC | 13.0 | 16.3 | 15.2 | 16.7 | Occurred in multiple place |
| | | | | | | | | (S₆ Φ1.4 cm and others) |
| 5 | Female | 84 | LC | 18.9 | 19.2 | 20.4 | 23.0 | S₈ Φ1.0 cm |
| 6 | Female | 67 | CH | 15.5 | 12.1 | 8.7 | 6.1 | S₈ Φ1.1 cm |
| 7 | Female | 58 | LC | 8.1 | 5.4 | | 5.1 | S₈/S₅ Φ1.0 cm |

AFP: Measured by Chemilumi ACS-AFP method

TABLE 2 shows the transition of AFP values in GM-administered HCC patients. Among all 7(seven) cases, one case was developed from chronic hepatitis, and the remaining 6(six) cases were developed from liver cirrhosis. On the whole, the elevation of AFP values was suppressed. Statistically, no significant difference between each month and 0M was confirmed.

All the liver cirrhosis cases indicated in TABLE 1 and TABLE 2 belonged to class A according to the Child-Pugh classification. Meanwhile, in the TABLE 1 and TABLE 2, “CH” and “LC” mean chronic hepatitis and liver cirrhosis, respectively, and “Occurred in multiple places” means the existence of 2 or more tumors.

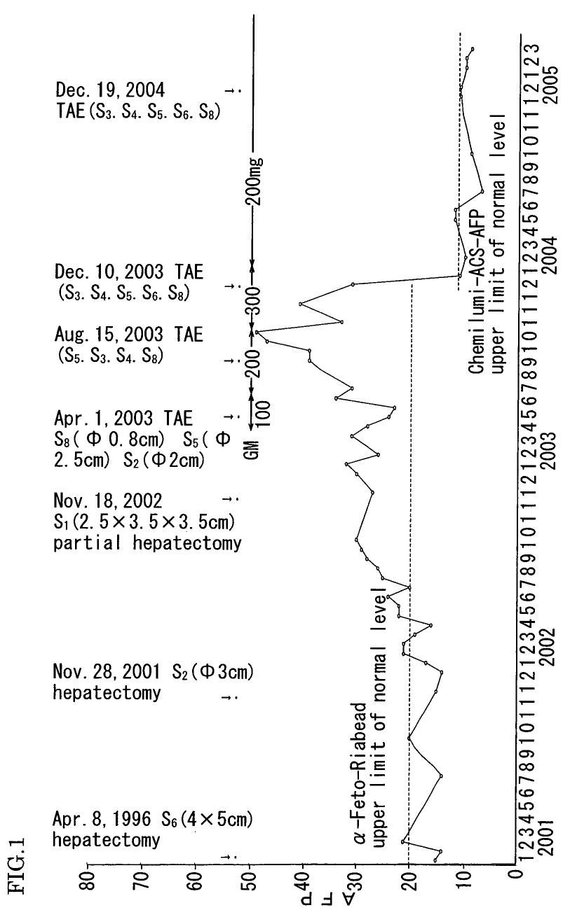

In TABLE 2, the first case is a 72-year-old male and who currently has HCC, 0.8-2.0 cm in diameter in S₁, S₂, S₄, S₆, S₇, and S₈ regions, and this case belongs to class A according to the Child-Pugh classification. In 1996, segmental resection of the liver(hepatectomy) was performed in HCC, 4×5 cm in diameter in the S₆ region. Subsequently, in November 2001, HCC, 3 cm in diameter, recurred in the S₂ region, and hepatectomy was performed. After that, in November 2002, HCC recurred in the S₁ region, and partial hepatectomy of 2.5×3.5×3.5 cm was performed. In April 2003, TAE was conducted against recurrent tumors in the S₂, S₅, and S₈ regions. And in June 2003, administration of 100 mg of GM was started. In August 2003, TAE was conducted against tumors mainly in the S₃, S₄, S₅, and S₈ regions, and the dose of GM was increased to 200-300 mg. After another TAE in December 2003, using this opportunity of hospitalization, TAE was conducted in viable HCC, 0.8 cm in diameter mainly in the S₃, S₄, S₅, S₆, and S₈ regions, though the AFP value had been below the normal limit for one year and TAE was not required at that time. Currently, AFP value of this case remains 9.0 that is below the standard level, and the general state is so satisfactory that the patient is leading a normal daily life. FIG. 1 is a diagram illustrating the transition of AFP values when gabexate mesilate is administered. In this diagram, the ordinate and abscissa indicate AFP values and dates, respectively.

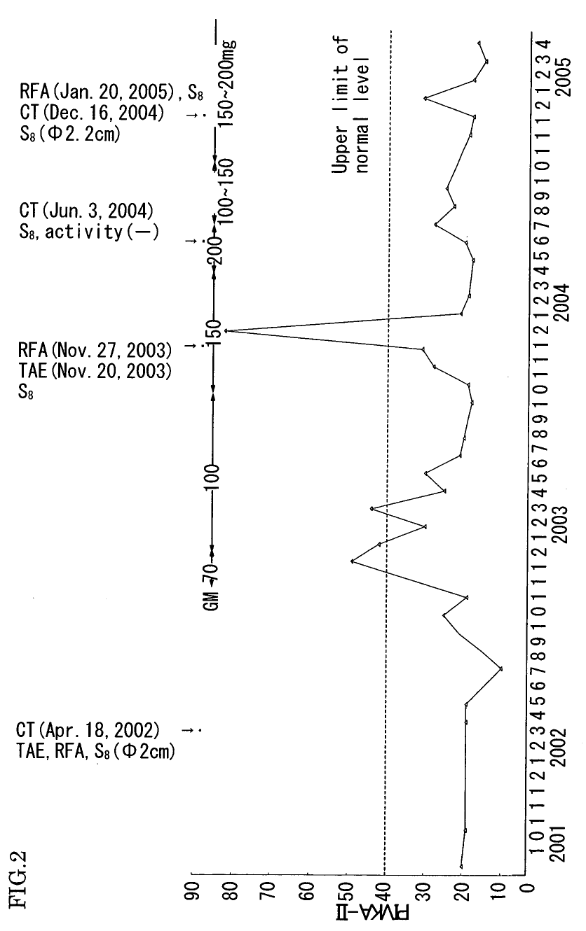

In TABLE 2, the second case is a 64-year-old female with type C liver cirrhosis, which belongs to class A according to the Child-Pugh classification. In this case, an abnormally high PIVKA-II value was observed at first, but recently, only an abnormal AFP value is observed. In April 2002, HCC, 2 cm in diameter in the S₈ region, was confirmed on contrast CT, therefore TAE and RFA were conducted. Then, administration of 70-160 mg of GM was started. Since a sharp rise of suppressed PIVKA-II values was observed, TAE and RFA were conducted again in the S₈ region in November 2003. Subsequently, GM was increased to 200 mg, and elevation of PIVKA-II and AFP values was not observed, and HCC, 2.2 cm in diameter in the S₈ region, was below normal limit markers (AFP and PIVKA-II) but seemed viable on contrast CT, therefore only RFA was conducted again after 14 months, and thereby the course of the disease remains excellent. FIG. 2 is a diagram illustrating the transition of PIVKA-II values when gabexate mesilate was administered. In this diagram, the ordinate and abscissa indicate PIVKA-II values and dates, respectively.

In TABLE 2, hepato cellular carcinoma, 1-1.5 cm in diameter in the S₈ region just beneath the dome could not be excluded in the 3rd case. Chronic pancreatitis was also confirmed, and 20 mg of GM had been administered. AFP values indicate the transition before diagnosis by contrast CT. In this patient, HCC was roughly diagnosed by contrast CT, but the AFP value was 8-9. Overseas travel for 2 weeks was permitted because of the patient's strong desire. After the patient's return from abroad, HCC was diagnosed by the elevated AFP value and on contrast CT. Also, an enlarged hepato cellular carcinoma was confirmed, therefore GM administration was continued after conducting TAE (epirubicin 20 mg, lipiodol 2 mg, and shredded spongel), and currently the course of the disease is excellent.

In TABLE 2, the 4th case is a patient (76-year-old male) with a mucous tumor and allergy, and the case is type C liver cirrhosis that belongs to class A according to the Child-Pugh classification and had been strongly suspected to have HCC by contrast CT for some time. GM dosage was reduced to 75-100 mg because of his allergy, but a sudden upward trend of AFP values was presented, and HCC was confirmed by follow-up CT, therefore 200 mg of GM was administered immediately after TAE was completed. Currently, the AFP value, which once rose up to 70, is around 7, and the course of the disease has been excellent for as long as 6 months.

The 5th case is an 84-year-old female, a case of elevated AFP values and hepato cellular carcinoma, 1.0 cm in diameter in the S₈ region, were confirmed after an approximately 10-year course of LC (C). The 6th case is a 67-year-old female, a case in which hepato cellular carcinoma, 1.1 cm in diameter in the S₈ region, was found by echo and contrast CT exams after an approximately 25-year course of chronic hepatitis C. The 7th case is a 58-year-old female, a case in which hepatic cancer, 1.0 cm in diameter in the S₈/S₅ regions, was similarly found by echo and contrast CT exams after an approximately 25-year course of chronic hepatitis C.

TABLE 3A, TABLE 3B, TABLE 3C and TABLE 3D indicate the transitions of AFP values, increase and decrease in AFP values, average AFP values, and standard deviations of AFP, in the GM-nonadministered HCC group, respectively, and TABLE 4A, TABLE 4B, TABLE 4C and TABLE 4D indicate the transitions of AFP values, increase and decrease in AFP values, average AFP values, and standard deviations of AFP, in the GM-administered HCC group with abnormal AFP values, respectively.

TABLE 5 and TABLE 6 indicate significant differences between 0M and each month in the GM-nonadministered HCC group and in the GM-administered HCC group with abnormal AFP values, respectively.

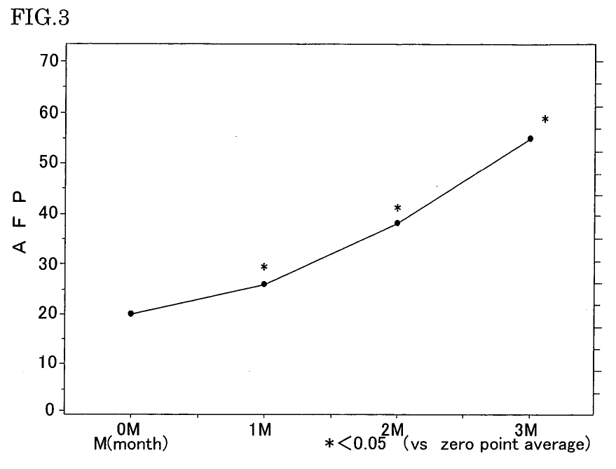

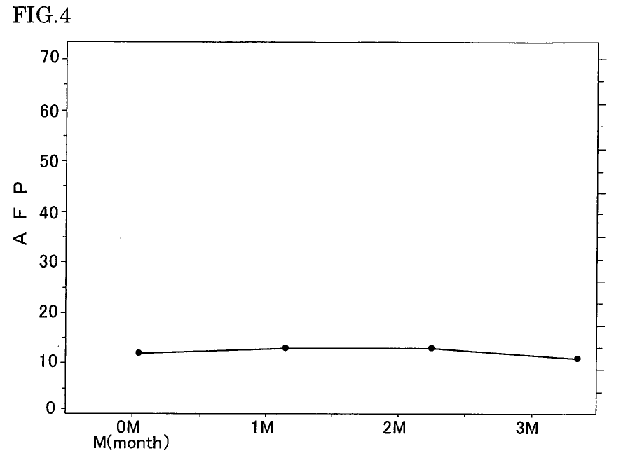

Statistically significant differences were clearly confirmed in the GM-nonadministered HCC group, but not in other group. FIG. 3 and FIG. 4 are diagrams illustrating the average AFP values and standard deviations of AFP in the GM-nonadministered HCC group and in the GM-administered HCC group with abnormal AFP values, respectively.

TABLE 3A

| | Sex | Age | Diagnosis | 0 M | 1 M | 2 M | 3 M |
|---|---|---|---|---|---|---|---|
| 1 | Female | 71 | CH | 17.4 | 30.5 | 48.7 | 56.7 |
| 2 | Female | 74 | LC | 23.6 | 28.5 | 30.6 | 34.1 |
| 3 | Female | 69 | LC | 14.4 | 30.8 | 42.5 | 66.9 |
| 4 | Male | 63 | LC | 43.0 | | 62.7 | 85.0 |
| 5 | Male | 53 | LC | 18.1 | 34.0 | 48.7 | |
| 6 | Male | 52 | LC | 13.7 | 15.3 | 20.6 | 25.2 |
| 7 | Male | 63 | LC | 12.9 | | 25.2 | 50.9 |
| 8 | Male | 67 | LC | 18.7 | 19.8 | 29.5 | 72.2 |

| | | | | |
|---|---|---|---|---|
| Average | 20.2 | 26.5 | 38.6 | 55.9 |

TABLE 3B

| | 0 M | Δ 1 M | Δ 2 M | Δ 3 M |
|---|---|---|---|---|
| 1 | 17.4 | 13.1 | 31.3 | 39.3 |
| 2 | 23.6 | 4.9 | 7 | 10.5 |
| 3 | 14.4 | 16.4 | 28.1 | 52.5 |
| 4 | 43.0 | | 19.7 | 42 |
| 5 | 18.1 | 15.9 | 30.6 | |
| 6 | 13.7 | 1.6 | 6.9 | 11.5 |
| 7 | 12.9 | | 12.3 | 38 |
| 8 | 18.7 | 1.1 | 10.8 | 53.5 |

| | | | |
|---|---|---|---|
| Average | 8.833333 | 18.3375 | 35.32857 |

TABLE 3C

| | | Standard | | | |
|---|---|---|---|---|---|
| | Average | deviation | The number of cases | Minimum | Maximum |
| 0 M | 20.2 | 9.8 | 8 | 12.9 | 43.0 |
| 1 M | 26.5 | 7.3 | 6 | 15.3 | 34.0 |
| 2 M | 38.6 | 14.4 | 8 | 20.6 | 62.7 |
| 3 M | 55.9 | 21.1 | 7 | 25.2 | 85.0 |

TABLE 3D

| | |
|---|---|
| 0 M | 20.2 ± 9.8 |
| 1 M | 26.5 ± 7.3 |
| 2 M | 38.6 ± 14.4 |
| 3 M | 55.9 ± 21.1 |

TABLE 4A

| | Sex | Age | Diagnosis | 0 M | 1 M | 2 M | 3 M |
|---|---|---|---|---|---|---|---|
| 1 | Male | 72 | LC | 7.2 | 5.9 | 5.8 | 4.0 |
| 2 | Female | 64 | LC | 8.5 | 12.1 | 11.6 | 13.6 |
| 3 | Male | 77 | LC | 6.5 | 8.9 | 6.2 | 6.7 |
| 4 | Male | 76 | LC | 13.0 | 16.3 | 15.2 | 16.7 |
| 5 | Female | 84 | LC | 18.9 | 19.2 | 20.4 | 23.0 |
| 6 | Female | 67 | CH | 15.5 | 12.1 | 8.7 | 6.1 |
| 7 | Female | 58 | LC | 8.1 | 5.4 | | 5.1 |

| | | | | |
|---|---|---|---|---|
| Average | 11.1 | 11.4 | 11.3 | 10.7 |

TABLE 4B

| | 0 M | Δ 1 M | Δ 2 M | Δ 3 M |
|---|---|---|---|---|
| 1 | 7.2 | −1.3 | −1.4 | −3.2 |
| 2 | 8.5 | 3.6 | 3.1 | 5.1 |
| 3 | 6.5 | 2.4 | −0.3 | 0.2 |
| 4 | 13.0 | 3.3 | 2.2 | 3.7 |
| 5 | 18.9 | 0.3 | 1.5 | 4.1 |
| 6 | 15.5 | −3.4 | −6.8 | −9.4 |
| 7 | 8.1 | −2.7 | | −3 |

| | | | |
|---|---|---|---|
| Average | 0.314286 | −0.28333 | −0.35714 |

TABLE 4C

| | | Standard | The number | | |
|---|---|---|---|---|---|
| | Average | deviation | of cases | Minimum | Maximum |
| 0 M | 11.1 | 4.8 | 7 | 6.5 | 18.9 |
| 1 M | 11.4 | 5.1 | 7 | 5.4 | 19.2 |
| 2 M | 11.3 | 5.7 | 6 | 5.8 | 20.4 |
| 3 M | 10.7 | 7.2 | 7 | 4.0 | 23.0 |

TABLE 4D

| | |
|---|---|
| 0 M | 11.1 ± 4.8 |
| 1 M | 11.4 ± 5.1 |
| 2 M | 11.3 ± 5.7 |
| 3 M | 10.7 ± 7.2 |

TABLE 5

| | | Difference from | Standard | | Significant |
|---|---|---|---|---|---|
| | Average | the value at 0M | deviation | p value | difference |
| 0 M | 20.2 | | | | |
| 1 M | 26.5 | 8.9 | 7.1 | 0.0277 | recognized |
| 2 M | 38.6 | 18.3 | 10.5 | 0.0117 | recognized |
| 3 M | 55.9 | 35.3 | 17.7 | 0.0180 | recognized |

TABLE 6

| | | Difference from | Standard | | Significant |
|---|---|---|---|---|---|
| | Average | the value at 0M | deviation | p value | difference |
| 0 M | 11.1 | | | | |
| 1 M | 11.4 | 0.3 | 2.9 | 0.7353 | none |
| 2 M | 11.3 | −0.3 | 3.6 | 0.7532 | none |
| 3 M | 10.7 | −0.4 | 5.2 | 0.7353 | none |

Side effects of GM used in the present invention already include shock, anaphylaxis, bleeding tendency, granulocytopenia, fall in blood pressure, elevation of GOT and GPT, facial flushing and the like; among 22 cases including pancreatic disease cases, shock: 0 case, anaphylaxis: 3 cases, bleeding tendency: 0 case, slight granulocytopenia: 22 cases, significant fall in blood pressure: 0 case, elevation of GOT and GPT: 2 cases, and withdrawal: 0.

As described above, gabexate mesilate used in the present invention is originally a drug applied to pancreatitis and DIC (disseminated intravascular coagulation), but has proved very effective in HCC treatment. In fact, blood levels of tumor markers such as AFP and PIVKA-II dropped, or their elevation was suppressed by administering drugs, which are gabexate mesilate as an active ingredient, to HCC patients.

In general, AFP and PIVKA-II values are considered to represent cancer viability in hepato cellular carcinoma cases. GM is considered to suppress the viability, since GM suppresses AFP and PIVKA-II values. GM in the amount of 100-300 mg dissolved in 150-300 ml of physiological saline or of 5% glucose was administered by intravenous drip infusion.

In single administration, it was considered effective if suppression of tumor markers was observed in one month. As described above, GM administration after TAE was conducted 10 days after TAE was completed. And GM was considered effective if suppression was observed 4 months after TAE was completed.

Concerning the GM administration method to HCC, it is possible to follow the course of early hepato cellular carcinoma with single GM administration. Against mid-stage hepato cellular carcinoma, TAE is conducted first, followed by GM administration around 10 days after TAE as described above, and another TAE and then GM treatment will be conducted when suppression is insufficient. In cases where GM becomes ineffective, it can be effective again by conducting another TAE and then GM treatment. In general, it becomes impossible to insert a catheter after conducting TAE 8(eight) times because of the damage to the arterial intima, and thereby the survival rate may be lowered. Therefore, extending TAE intervals, by GM treatment, is considered to improve the survival rate.

As described above, the number of times of effective GM administrations after TAE was 6(six) in the cases of TABLE 2, and 7(seven) in the single GM-administered cases including 4(four) cases with abnormal AFP values of unknown etiology (early hepato cellular carcinoma or during regeneration after hepatic damage).

Meanwhile, as shown in FIG. 1 and FIG. 2, a drug gabexate mesilate as an active ingredient, administered after TAE, suppressed the elevation of AFP values for a prolonged period. In addition, there is a possibility of improving the survival time, since concomitant use with TAE strongly suppressed HCC and exerted effects to extend the TAE intervals.

A drug containing gabexate mesilate can be administered noninvasively with little or no pain to patients. Also, there is a possibility of suppressing canceration of precancerous lesion.

1. A method for treating cancer which comprises administering gabexate mesilate to a cancer patient.

2. The method for treating cancer according to claim 1, wherein the cancer is hepato cellular carcinoma.
